(19) United States
(12) Patent Application Publication
(10) Pub. No.: US 2003/0114930 A1
(43) Pub. Date: Jun. 19, 2003

(54) Apparatus and method to stabilize and repair an intervertebral disc

(76) Inventors: Kit Yeng Lim, College Station, TX (US); Jeff Moehlenbruck, Austin, TX (US)

Correspondence Address: Timothy L. Scott, Senior Intellectual Property Counsel, SULZER MEDICA USA INC., 3 East Greenway Plaza, Suite 1600, Houston, TX, 77046, US

(21) Appl. No.: 10/023,911

(22) Filed: Dec. 18, 2001

(51) Int. Cl. A61F 2/44

(52) U.S. Cl. 623/17.11

(57) ABSTRACT

A method and apparatus for sealing a defect in an annulus is provided herein. A plug made from a biodegradable or biocompatible polymer or a combination thereof can be used to seal the defect. The plug can include a cage with a sealed end and at least one retaining member on the outer surface. In one embodiment, the plug includes a hollow bore that is filled with a matrix of a growth-promoting compound. An aperture formed on the outer surface of the plug provides a pathway for surrounding cells to access the matrix. In another embodiment, a cap with a slot is provided at a second end of the plug. In still another embodiment, the cap is provided with no slot therein. Further, the plug may be inserted into the defect of the annulus by a tool. Additionally, a method using the plug is provided to seal the defect.

### BACKGROUND OF THE INVENTION

The present invention generally relates to an apparatus and method to stabilize and repair an intervertebral disc.

### DESCRIPTION OF THE RELATED ART

The human spinal column is formed from a plurality of articulating bony vertebrae, which are separated from and connected to one another by 23 cartilaginous intervertebral discs. Intervertebral discs (hereinafter also “discs”), separate and provide structural support to the vertebrae and distribute load forces applied to the spinal column. The discs accomplish this by acting as a cushion between adjacent vertebrae, resisting axial compression along the spinal column, but permitting limited flexion, extension, and rotation between vertebrae. The discs thus allow for the characteristic smooth motion and flexibility of the healthy human spine. FIG. 1 illustrates a portion of the spinal column 5. The intervertebral disc 10 comprises a central, inner portion of soft, gelatinous, mucoid material, the nucleus pulposus (not shown, hereinafter “nucleus”), which is peripherally surrounded by an annular ring of lamellae of tough, fibrous material, the annulus fibrosus 15 (hereinafter “annulus”). The nucleus and the annulus 15 together are bounded cranially and caudally by end-plates 12 of the adjacent vertebrae. These end-plates 12 are composed of a thin layer of hyaline cartilage and attach at their peripheries to the lamellae of the inner portions of the annulus. The lamellae of the outer portions of the annulus 15 attach directly to the bone at the outer edges of the adjacent vertebrae. The nucleus has no distinct attachments to the end-plates 12 but connects loosely to the fibrous collagenous network of the inner annulus.

In the human adult, the nucleus contains rounded chondrocyte-like cells which produce collagen fibrils (primarily Type II collagen, but also Types IX, XI, and others) and large molecules of negatively charged, sulfated proteoglycans. The collagenous and associated protein components of the extracellular matrix in the nucleus comprise a scaffold for cellular attachment, migration, and proliferation. The proteoglycan component of the nucleus produces a high fixed charge density, which attracts water to form a hydrated gel. By functioning as a hydraulic fluid that assists the dispersion of spinal forces, the nucleus pulposus plays a central role in maintaining intervertebral disc hydrodynamic function. In the normal healthy nucleus, water comprises between 80-90% of the total weight. The large molecular weight proteoglycans, which are contained within the nucleus by the annulus and by the vertebral end-plates, attract water into the nucleus through sieve-like pores in the end-plates. The resulting osmotic pressure within each disc tends to expand it axially, driving adjacent vertebrae further apart. Counter-acting these osmotic forces are compressive loads on the intervertebral discs 10, which tend to drive water out of the nucleus and reduce disc height. Osmotically-driven exchange of water and solutes between discs and vertebrae from normal diurnal fluctuations facilitates chondrocyte nutrition and respiration within the discs. This nutritive function is critical to chondrocyte survival since the high pressures occurring in intervertebral discs do not allow the presence of blood capillaries; in fact intervertebral discs 10 are the largest avascular structures in the human body. The high water content in the nucleus is also important for absorbing high mechanical (shock) loads, and for hydrating the annulus 15 to maintain flexibility and strength, which in turn helps in resisting herniation of nucleus matter into or through the annulus under high loads.

The normal hydrodynamic functions of healthy discs as described above are compromised in degenerative disc disease (hereinafter “DDD”). DDD is caused by deterioration in the structure and function of intervertebral discs and is associated with aging and/or spinal trauma. Although the etiology of DDD is not well understood, one consistent change in degenerative discs is an overall decrease in proteoglycan content within the nucleus pulposus and the annulus fibrosus. The loss in proteoglycan content results in a concomitant loss of water content. Reduced hydration of disc structures, particularly the nucleus, can cause an increased amount of load to be transferred to the annulus. This load transfer onto an increasingly dehydrated annulus can cause tearing in the layers of the annulus or an annular defect 20 (see FIG.1), which may render the disc susceptible to herniation. Herniation usually results in extruded disc material impinging on the spinal cord or spinal roots, causing pain, weakness, and possible loss of motor functions. If the defect 20 is left untreated, the disc material can continue to press on the spinal nerves and cause pain to the patient. Additionally, loss of disc height can occur due to the extrusion of the nucleus through the defect 20. The extrusion decreases the nucleus' ability to separate and cushion the vertebrae. The loss of disc height also can lead to abrasion between adjacent vertebrae. Accelerated disc degeneration can also occur through further tearing of the annulus 15. Since the majority of disc tissue receives no blood supply, damaged or degenerated intervertebral disc tissue is extremely limited in its capacity for self-repair.

There are an estimated 5 million adults in the US who suffer from chronic low back and neck pain. The correlation between DDD and these spine-related pain syndromes is quite significant. Currently, medical personnel frequently use magnetic resonance imaging (MRI) to diagnose herniated discs. From the images, a physician can assess appropriate treatments discussed herein, infra. One such class of treatments is surgery for intervertebral disc herniation and chronic intervertebral disc degeneration, which may include discectomy (removal of protruding disc tissues) and/or spinal fusion (complete disc removal and bone fusion of the two adjacent vertebrae). Discectomy involves removing the protruding nucleus from the disc. While often effective in alleviating acute pain associated with disc material impinging upon the spinal cord, the loss of the hydraulic capacity of the removed nucleus material in a discectomy frequently leads to continued disc degeneration because a lesser amount of hydrated nucleus pulposus is available to cushion or counteract weight on the disc. Additionally, discectomy may cause loss of disc height and facet joint degeneration, wherein the articulating surfaces of the facets of an upper and lower vertebra abrade against each other.

Spinal fusion is an invasive, risky, and traumatic surgical procedure, which requires lengthy recovery times. Fusion is considered the treatment of last resort, reserved for patients with significant intractable pain and/or neurological deficits. A number of devices and methods have been developed for the replacement of degenerated or dislocated intervertebral discs in order to achieve spinal fusions. For example, autogenous grafts of dowel-shaped sections of bone are implanted between the vertebrae to cause bone growth across the intervertebral space, thereby fusing the adjacent vertebrae into one bone mass. The harvest of donor bone for the autogenous graft comes from another location, such as the hip, thus requiring a separate surgical procedure and resultant increases in complications and expense. An alternative source of bone for fusion surgery is cadaveric allograft bone, which eliminates the need for a second surgical procedure to harvest the allograft, along with the associated pain and discomfort for the patient. However, the use of allograft material raises the possibility of potential complications related to transmissible diseases or non-fusions related to poor graft incorporation, collapse or displacement. A more recent development in spine fusion surgery involves the use of Interbody Fusion (hereinafter “IBF”) cage devices which are fenestrated, threaded, cylindrical cages, constructed of titanium and/or other materials. The surgeon implants the IBF cages by screwing them into the interbody space between adjacent vertebrae. Bone fragments produced in preparing the vertebrae for the implantation are packed into the cages to promote bone growth into, through and around the cage.

Additional therapies for disc degeneration focus on nucleus replacement in the form of prostheses or biologic injectibles. U.S. Pat. No. 6,240,926 discloses therapy wherein tissue from the nucleus is removed, and the space is filled with a hybrid material, such as bioactive glass or polymer foam. This therapy is problematic because it can subject a patient to unnecessary removal of tissue from the nucleus. U.S. Pat. No. 6,224,630 discloses another type of therapy wherein an expandable, porous plug is implanted into an annular defect. The plug is then expanded by hydration. This therapy is problematic because improper hydration of the expandable material may cause the plug to fit incorrectly, and not be well secured in the defect. If the plug is not well secured in the defect, it may be displaced due to the pressure exerted on it by the nucleus.

Therefore, there is a need for an apparatus and method to stabilize and repair an intervertebral disc.

### SUMMARY OF THE INVENTION

The present invention relates generally to an apparatus and method to repair and seal a defect in an annulus of an intervertebral disc. The apparatus preferably comprises a plug that includes a biodegradable hollow member having at least one retaining member on an outer surface thereof, e.g., a thread or barbs. A biodegradable matrix formed of, e.g., a biocompatible, growth promoting scaffold matrix material can be disposed in the hollow member. The hollow member can be made from a polymer, and may include a cap at an end with a slot therein that is capable of mating with a tool for ease of surgical placement. Additionally, the plug can include a growth promoting compound. Preferably, the matrix includes or is made from the growth promoting compound, while the hollow member that preferably encases or surrounds the matrix is preferably made of a biodegradable material. The at least one retaining member can include at least one retaining ridge. Further, the hollow member may include at least one portal or aperture in the wall thereof to provide a contact of the disc structures with the matrix disposed therein.

In another embodiment, the plug comprises a biodegradable hollow member having an outer surface and a growth promoting matrix disposed therein. A cap may also be provided at an end of the hollow member. Additionally, at least one aperture providing communication between the outer surface of the hollow member and the matrix, and at least one retaining member on the outer surface of the hollow member can be provided. The growth promoting matrix is preferably chondro-inductive. The cap preferably further includes a slot therein for mating with a tool for ease of surgical implantation. The hollow member can be made from a polymer, while the matrix can be made from a growth-promoting compound. Further, the at least one retaining member provided on the outer surface of the cage can include at least one retaining ridge. Preferably, the outer surface of the hollow member has a plurality of retaining ridges, e.g., 2-5 circumferential threads, for gripping a defect. The retaining ridge preferably extends around the circumference of the hollow member.

A method of sealing a defect in an annulus of a human intervertebral disc provided herein includes providing a plug comprising a biodegradable hollow memberhaving at least one retaining member on an outer surface thereof and a growth promoting matrix coupled thereto; and inserting the plug into the defect of the annulus of the intervertebral disc. The hollow member of the plug preferably has a first end and a second end. The hollow member can have a sealing member at one end. Additionally, the hollow member can include a cap at an end, the cap having a slot therein for mating with a tool. The plug can include growth promoting compounds. Further, the hollow member can be made from a polymer and can contain growth promoting compounds. The matrix can be chondro-inductive. The at least one retaining member can further include at least one retaining ridge. At least one portal extending there-through to the matrix of the hollow member is also provided. Inserting the plug into the defect can also include inserting, rotating, screwing, threading, or tapping the plug so as to position it within the defect. Additionally, a tool is typically used to insert the plug into the defect.

### BRIEF DESCRIPTION OF THE DRAWINGS

A more particular description of the invention summarized above may be had by reference to certain embodiments thereof which are illustrated in the appended drawings. It is to be noted, however, that the appended drawings illustrate only typical embodiments of this invention and are therefore not to be considered limiting of its scope, as the invention may admit to other equally effective embodiments.

FIG. 1 illustrates a portion of spinal column.

FIG. 2A is a side view of a plug having retaining members on the outer surface.

FIG. 2B is an angle view of a further embodiment of plug having a cap with a slot disposed at an end thereof, and an aperture which extends through an outer surface.

FIG. 2C is an end view of the plug shown in FIG. 2A along line 2C-2C that is filled with a matrix.

FIG. 3 is a schematic view of an embodiment of a plug positioned to be inserted into an annulus defect.

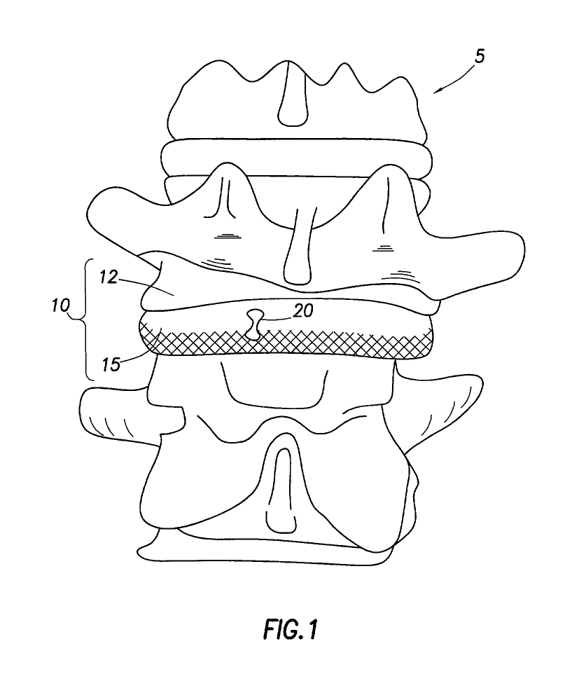

### DETAILED DESCRIPTION OF THE PREFERRED EMBODIMENT

As discussed above, an apparatus of the invention may be provided as a plug comprising a biodegradable hollow member (e.g., a cage) having a retaining member, e.g., a ridge or series of ridges such as threads. The retaining member is preferably, though not necessarily, integral with the hollow member. The hollow member preferably comprises a biodegradable material, e.g., a biodegradable polymer, which is also preferably biocompatible. As used herein, the term “plug” means a member, preferably an elongated member, having a size and shape adapted to fit into a defect. A plug preferably comprises a cage having at least one retaining member. As used herein, the term “cage” means a portion of the plug, preferably an outer portion of the plug. The cage can be solid or have a totally or partially hollow center, with either a single cylindrical bore running through the cage, or a plurality of parallel cylindrical bores extending lengthwise from one end of the cage to another end of the cage. As used herein, the term “retaining member” means any structure that can retain the plug in place once the plug is inserted into the annulus. In one embodiment, the retaining members comprise multiple threads along the outer surface of the cage. The retaining members can either be integrated with the cage, e.g., as threads, or coupled to the outer surface of the cage, e.g., as circumferentially disposed clamps.

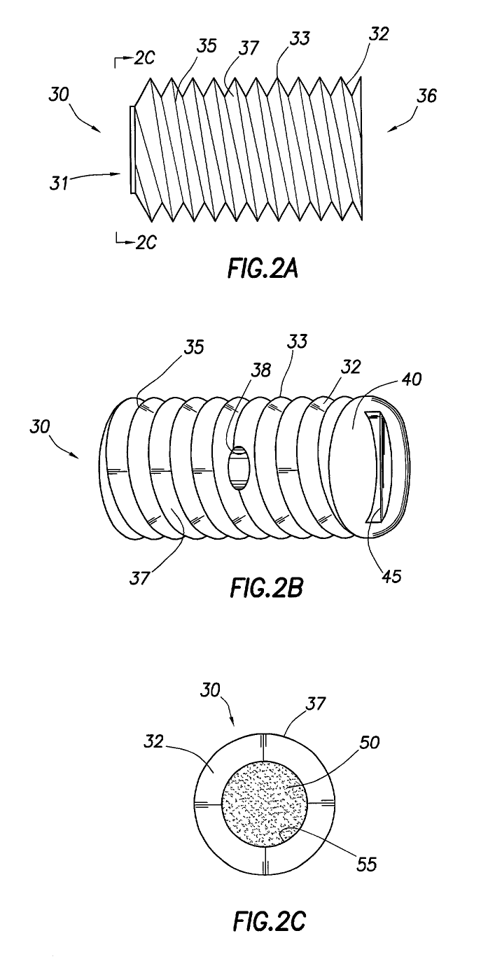

FIG. 2A is a side view of a plug 30 having retaining members 35 on the outer surface. The plug 30 is shown as being cylindrically shaped and is defined by an outer covering or cage 32. The plug 30 is preferably made of a solid material, although woven, braided or other designs may be used. Cage 32 is preferably made from a biodegradable polymer that is preferably also biocompatible. The polymer may be natural or synthetic. The term “biodegradable” as used herein refers to a material that breaks down in the body. The term “biocompatible” as used herein refers to a material that is not harmful to and does not cause an undesirable immunological response in a body, e.g., a human being. While certain materials are biocompatible, e.g., titanium, they are not biodegradable, because they do not break down in the body, and thus remain present in the body even after long periods of time.

Preferably, the portion of the plug that is biodegradable, e.g., the cage surrounding the matrix, comprises a biodegradable polymer such as, but not limited to, poly(L-lactides) (PLLA), poly(lactide-co-glycolides) (PLGA), polylactides (PLA), polyglycolic acids (PGA), polycaprolactones (PCL), polycarbonates, polyamides, polyanhydrides, polyamino acids, polyortho esters, polyacetals, polycyanoacrylates, degradable polyurethanes, albumin, collagen, elastin, reticulin, synthetic polyamino acids, prolamines, polysaccharides such as alginate, heparin, other biodegradable polymers of sugar units, and combinations thereof. Biocompatible polymers that may not be biodegradable include, but are not limited to, various polyacrylates, ethylene-vinyl acetates (and other acyl-substituted cellulose acetates), polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, and combinations thereof.

In a further embodiment, the cage 32 may have one or more growth promoting compounds disposed thereon (e.g., as part of a coating) or therein (e.g., as part of a matrix) to stimulate growth of tissue surrounding the plug 30 after it is inserted into an annulus 15. Suitable growth promoting compounds may include, but are not limited to, angiogenic factors, immune system suppressors such as anti-inflammatory agents, antibiotics, living cells, cell-binding proteins and peptides, and similar compounds. Growth promoting compounds can also include growth factors that may induce cartilage growth (chondro-inductive), such as polypeptides of the TGF-βsuperfamily, which can include TGF-β1, TGF-β2, and TGF-β3, GDF-5 (MP52), BMPs (bone morphogenetic proteins), GFm (Applicant's proprietary mixture) and other similar compounds. Advantageously, in accordance with this embodiment, the growth promoting compound(s) can stimulate growth of the surrounding tissues, so that the plug 30 can “permanently” seal the defect 20, even after the hollow support member, e.g., cage, has been degraded and is no longer present. That is, sealing the defect 20 with the surrounding tissues, whereby the plug can be integrated with surrounding tissues and seal the defect.

As shown in FIG. 2A, the cage 32 preferably includes opposing ends, namely, a first end 31 and a second end 36. The cage 32 is constructed and arranged with at least one retaining member 35 thereon. As seen in FIG. 2A, the retaining members 35 for cage 32 may comprise threads. Although the retaining members 35 are shown to be disposed at an angle in relation to the normal (vertical), the angle is not required. In particular, the retaining member 35 includes at least one retaining means such as retaining ridge 33. In use, the retaining member 35 and retaining ridge 33 secure the plug 30 once it is inserted into the annulus 15. In addition, the retaining member 35 and the retaining ridge 33 also resist forces that can cause the plug 30 to pop out. Preferably, the cage 32 has a plurality of retaining members 35 and retaining ridges 33 thereon. Although the retaining members 35 are depicted as retaining ridges 33, one of ordinary skill in the art will readily appreciate the present invention is not so limited. In fact, the retaining members can be embodied by any mechanical means that can retain the plug 30 in place once inserted into the annulus 15. Although the plug 30 is shown in this and subsequent figures as cylindrical in shape, the plug can be any size or shape so long as it can fit into the defect 20. The plug 30 preferably includes a retaining member 35.

The plug 30 provided herein is designed to be inserted into a defect 20 and prevents and/or inhibits further extrusion of nucleus from the annulus 15. The plug 30 may be inserted into the defect 20 by an insertion tool such as a hemostat (clamp-like instrument), a catheter (hollow flexible tube), pliers, or similar surgical tools. As persons of skill in the art will appreciate, the insertion tool should be of a kind that does not damage the plug 30 when used. If the catheter is used, the plug 30 may be placed at an end of the catheter and inserted into the annulus 15. Once inserted, in accordance with one embodiment of the present invention, the retaining members 35 will keep plug 30 from popping out or being otherwise dislodged from the annulus 15. If the hemostat is used, the plug can be mechanically held by the hemostat and inserted into the annulus 15. Again, the retaining members 35 retain the plug 30 in the annulus 15 when the hemostat mechanically releases the plug.

FIG. 2B is an angle view of a further embodiment of plug 30 having a cap 40 with a slot 45 disposed at an end, and an aperture 38 which extends through an outer surface 37 to an inner bore 55 of the plug 30. In a preferred embodiment, the cage 32 comprises a hollow bore 55 (FIG. 2C) and has at least one aperture 38 connecting the hollow center 55 with the outer surface 37. Alternatively, instead of a single bore 55, the cage can have multiple hollow openings, as described elsewhere, which can resemble longitudinal capillaries. Similarly, multiple apertures may be provided for communication between the outer surface 37 and the interior of the plug 30. When the cage 32 is hollow, at least one end is preferably sealed to prevent nucleus from extruding there-through when in use. The aperture 38 provides a pathway to a matrix 50 (FIG. 2C) that can be disposed within the hollow center 55 of the cage 32. In a preferred embodiment of the present invention, the matrix 50 contains a growth promoting compound (discussed above) in order to stimulate growth of cells in the compound and cells of the tissues surrounding the cage 32. As cells in the bore 55 of the plug 30 and in the surrounding tissues grow and propagate, they help to integrate and secure the plug 30 within the annulus 15. When the plug 30 is integrated with the annulus 15, the plug can provide a permanent seal of the defect 20.

The aperture 38 enables migration of cells from the surrounding tissues into the matrix 50 and allows diffusion of growth factors from the matrix 50 into the surrounding tissues, leading to more secure placement of the plug 30 in the annulus 15. Although only one apeture 38 is shown in FIG. 2B, as many apertures as needed can be formed on the outer surface 37 of the cage 32. The cap 40 is coupled to the second end 36 of the plug 30 and has a slot 45 formed therein. Although the cap 40 is preferably coupled to the plug 30, the cap may be integrated with the plug. The slot 45 may be any female recess that is capable of receiving a corresponding male portion of a manipulating tool. In accordance with one embodiment of the present invention, the plug 30 is inserted into the defect 20 using the manipulating tool. The manipulating tool may be a slotted, Phillips or hex shaped screwdriver, or any other tool capable of inserting the plug 30 into the defect 20. Additionally, the plug 30 may be rotated, threaded, screwed or manipulated in any other manner that allows the plug 30 and its retaining member 35 to enter the defect 20.

In an alternative embodiment, the cap 40 is flat with no slot 45. In accordance with this embodiment, plug 30 is inserted into the defect 20 by tapping the flat surface. The plug 30 can be tapped into the annulus 15 where the annulus 15 contains fibrous tissues. The fibrous tissues allow the annulus 15 to slightly stretch to receive the plug 30. Thereafter, the annulus 15 returns to its original shape. Advantageously, in accordance with the present invention, the retaining member 35 with its retaining ridges 33 retains the plug 30 in the defect, thereby preventing further degeneration of the disc. The plug 30 may be tapped into the defect 20 with a tool having a flat surface. The tool may be a small hammer or any other tool that can tap the plug 30 into the defect 20.

FIG. 2C is an end view of the plug 30 shown in FIG. 2A along line 2C-2C, depicting bore 55 filled with a matrix 50. The sealing member (if plug is hollow) of first end 31 is removed for illustrative purposes to show the matrix 50. Alternatively, the bore 55 may be open at end 31 to allow migration of cells into the matrix 50 and diffusion of growth factors from matrix 50 into the surrounding annulus and nucleus. The cage 32 includes a hollow bore 55, wherein the bore can be loaded or filled with the matrix 50. The bore 55 can be manufactured with varying sizes to vary structural support of the plug 30, and vary the amount of the matrix 50 it can hold. If more structural support is required of the plug 30, the diameter of the bore 55 is decreased allowing for a decrease in the inner diameter of the plug and a corresponding increase in the wall thickness for increased strength. If more matrix 50 is required then the diameter of the hollow center 55 is increased allowing for more volume for the matrix.

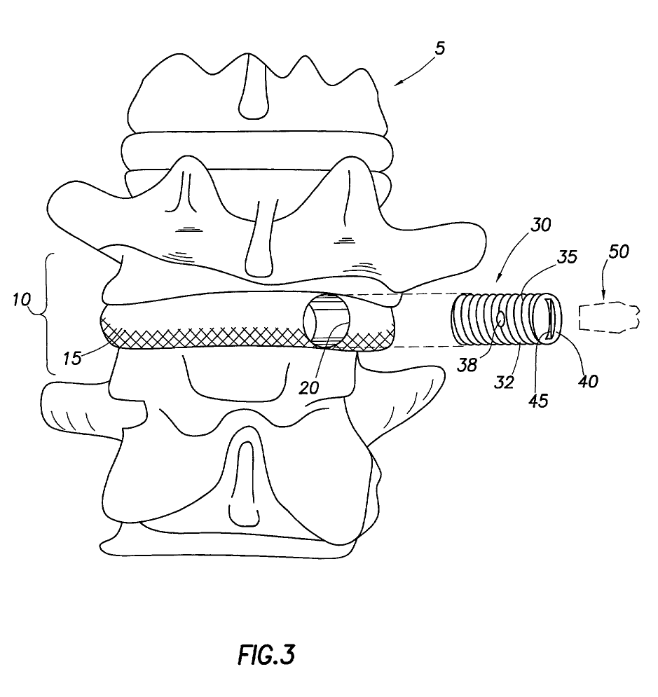

FIG. 3 is a schematic view of an embodiment of a plug 30 positioned to be inserted into a defect 20 of an annulus 15. A portion of spinal column 5 is shown having an intervertebral disc 10 therein, which includes the annulus 15 having a defect 20 therein. The defect 20 may be caused by degeneration of the annulus 15 due to herniation or injury. The plug 30 is shown to comprise retaining members 35, aperture 38 thereon, and a cap 40 with slot 45 at an end. The plug 30 is also shown in position to be inserted into the defect 20 with a manipulating tool 50 (shown in phantom as a screwdriver tip).

In operation, the annular defect 20 can be located using magnetic resonance imaging or other conventional diagnostic techniques. Using such non-invasive and/or minimally invasive techniques and known measuring devices, the defect's 20 approximate size and shape can be determined. The plug 30 can be made to have similar size and shape as the defect 20. Preferentially, the defect 20 can be surgically altered to conform to the size and shape of a pre-manufactured plug 30.

The plug 30 and the defect 20 can be “custom fitted” to provide a good fit in the annulus 15. That is, the plug 30 and defect 20 can be formed to fit each other. Then, the plug 30 can be inserted into the defect 20 by a tool such as a catheter or a hemostat. In an alternative embodiment, the plug 30 can be inserted by screwing it into the annulus 15 over a predetermined distance by using a tool 50 such as a screwdriver. Additionally, in another embodiment, the plug 30 is inserted by tapping the plug into the defect 20. Although tools have been described to insert the plug 30 into the defect 20, the surgeon may also insert the plug manually.

In accordance with certain embodiments of the present invention, after the plug 30 is inserted into the defect 20 of annulus 15, the cells surrounding the defect 20 can proliferate in and around the plug 30. As noted above, cells can proliferate around the cage 32 due to the matrix 50 and its growth promoting compounds. The cage 32 provides a stable platform wherein the cells can attach and grow, and the aperture(s) 38 allow access for the cells to the growth promoting compounds in the matrix 50. When a biodegradable polymer is used with plug 30, then after a certain period of time, the biodegradable polymer of the cage 32 should degrade, leaving the cells that have grown in place. Once growth is completed, the defect 20 should be permanently sealed thereby, preventing further degeneration of the disc. The plug 30 may be used as a stand-alone treatment or in conjunction with treatments to retard disc degeneration such as discectomy.

While the foregoing is directed to embodiments of the present invention, other and further embodiments of the invention may be devised without departing from the basic scope thereof, and the scope thereof is determined by the claims that follow, including equivalents.

What is claimed is:

1. An apparatus for repairing a defect in an annulus comprising a plug having:
a biodegradable member having an outer surface and a bore;
a growth promoting matrix disposed in the biodegradable member; and
at least one retaining member on the outer surface of the biodegradable member.

2. The apparatus of claim 1, wherein the biodegradable member further comprises a cap at an end.

3. The apparatus of claim 1, wherein the biodegradable member further comprises a cap at an end, the cap having a slot therein for mating with a tool.

4. The apparatus of claim 1, wherein the biodegradable member is made from a polymer.

5. The apparatus of claim 1, wherein the biodegradable member is made from a polymer selected from the group consisting of poly(L-lactides) (PLLA), poly(lactide-co-glycolides) (PLGA), polylactides (PLA), polyglycolic acids (PGA), polycaprolactones (PCL), polycarbonates, polyamides, polyanhydrides, polyamino acids, polyortho esters, polyacetals, polycyanoacrylates, degradable polyurethanes, albumin, collagen, elastin, reticulin, synthetic polyamino acids, prolamines, polysaccharides, alginate, heparin, biodegradable polymers of sugar units, and combinations thereof.

6. The apparatus of claim 1, wherein the growth promoting matrix includes a growth promoting compound.

7. The apparatus of claim 1, wherein the growth promoting matrix includes a growth promoting compound selected from the group consisting of growth factors, angiogenic factors, immune system suppressors, anti-inflammatory agents, antibiotics, living cells, cell-binding proteins and peptides, and combinations thereof.

8. The apparatus of claim 1, wherein the growth promoting matrix includes a growth factor selected from the group consisting of TGF-β1, TGF-β2, and TGF-β3, GDF-5, BMPs and GFm.

9. The apparatus of claim 1, wherein the at least one retaining member includes at least one retaining ridge.

10. The apparatus of claim 1, wherein the biodegradable hollow member includes at least one aperture providing communication between the outer surface and the bore.

11. An apparatus for repairing a defect in an annulus comprising a plug having:
a biodegradable member having an outer surface and a bore;
a growth promoting matrix disposed in the bore;
a cap at an end of the biodegradable member;
at least one aperture connecting the outer surface of the biodegradable member to the growth promoting matrix disposed in the bore; and
at least one retaining member on the outer surface of the biodegradable member.

12. The apparatus of claim 11, wherein the growth promoting matrix is chondro-inductive.

13. The apparatus of claim 11, wherein the cap has a slot therein for mating with a tool.

14. The apparatus of claim 11, wherein the biodegradable member is made from a polymer.

15. The apparatus of claim 11, wherein the biodegradable member is made from a polymer selected from the group consisting of poly(L-lactides) (PLLA), poly(lactide-co-glycolides) (PLGA), polylactides (PLA), polyglycolic acids (PGA), polycaprolactones (PCL), polycarbonates, polyamides, polyanhydrides, polyamino acids, polyortho esters, polyacetals, polycyanoacrylates, degradable polyurethanes, albumin, collagen, elastin, reticulin, synthetic polyamino acids, prolamines, polysaccharides, alginate, heparin, biodegradable polymers of sugar units, and combinations thereof.

16. The apparatus of claim 11, wherein the matrix comprises a growth promoting compound.

17. The apparatus of claim 12, wherein the matrix comprises a growth promoting compound selected from the group consisting of growth factors, angiogenic factors, immune system suppressors, anti-inflammatory agents, antibiotics, living cells, cell-binding proteins and peptides, and combinations thereof.

18. The apparatus of claim 11, wherein the at least one retaining member includes at least one retaining ridge.

19. A method of sealing a defect in an annulus of a human intervertebral disc comprising:
providing a plug comprising a biodegradable member having an outer surface, a growth promoting matrix, and at least one retaining member on the outer surface; and
inserting the plug into the defect of the annulus of an intervertebral disc.

20. The method of claim 19, wherein the biodegradable member has a first end and a second end.

21. The method of claim 19, wherein the growth promoting matrix comprises a growth promoting compound selected from the group consisting of growth factors, angiogenic factors, immune system suppressors, anti-inflammatory agents, antibiotics, living cells, cell-binding proteins and peptides, and combinations thereof.

22. The method of claim 19, wherein the biodegradable member has a sealing member at one end.

23. The method of claim 19, wherein the biodegradable member further comprises a cap at an end.

24. The method of claim 19, wherein the biodegradable member further comprises a cap at an end, and the cap has a slot therein for mating with a tool.

25. The method of claim 19, wherein the biodegradable member comprises a polymer.

26. The method of claim 19, wherein the biodegradable member is made from a polymer selected from the group consisting of poly(L-lactides) (PLLA), poly(lactide-co-glycolides) (PLGA), polylactides (PLA), polyglycolic acids (PGA), polycaprolactones (PCL), polycarbonates, polyamides, polyanhydrides, polyamino acids, polyortho esters, polyacetals, polycyanoacrylates, degradable polyurethanes, albumin, collagen, elastin, reticulin, synthetic polyamino acids, prolamines, polysaccharides, alginate, heparin, biodegradable polymers of sugar units, and combinations thereof.

27. The method of claim 19, wherein the biodegradable matrix is chondro-inductive.

28. The method of claim 19, wherein the matrix comprises a growth promoting compound selected from the group consisting of growth factors, angiogenic factors, immune system suppressors, anti-inflammatory agents, antibiotics, living cells, cell-binding proteins and peptides, and combinations thereof.

29. The method of claim 19, wherein the at least one retaining member comprises at least one retaining ridge.

30. The method of claim 19, wherein the biodegradable member comprises at least one aperture extending there-through to the growth promoting matrix.

31. The method of claim 19, whereby inserting the plug includes inserting, rotating, screwing, threading, or tapping the plug as to place the plug within the defect.

32. The method of claim 19, wherein inserting the plug is done using a tool.

33. The method of sealing a defect of claim 32, wherein the tool is selected from the group consisting of a hemostat, a catheter, pliers, a slotted screwdriver, a Phillips shaped screwdriver, a hex shaped screwdriver, and a hammer.
